(19) United States
(12) Patent Application Publication
(10) Pub. No.: US 2008/0161850 A1
(43) Pub. Date: Jul. 3, 2008

(54) SUTURE ANCHORING SYSTEM

(76) Inventors: William Bruce Weisenburgh, Maineville, OH (US); Michael S. Cropper, Edgewood, KY (US); Robert P. Gill, Mason, OH (US); David T. Martin, Milford, OH (US); Christopher J. Hess, Cincinnati, OH (US); James W. Voegele, Cincinnati, OH (US)

Correspondence Address: KING & SCHICKLI, PLLC, 247 NORTH BROADWAY, LEXINGTON, KY, 40507, US

(21) Appl. No.: 11/617,375

(22) Filed: Dec. 28, 2006

(51) Int. Cl. A61B 17/04

(52) U.S. Cl. 606/230; 606/232

(57) ABSTRACT

A first extruded member in joined to a molded member to form an integrated component. In one exemplary form, a surgical anchor is overmolded onto the end of an extruded suture to form a suture/anchor system.

### FIELD OF THE INVENTION

The invention relates generally to surgical fastening systems and more particularly to a system for anchoring sutures.

### BACKGROUND

Sutures are commonly used in the medical profession for holding areas of tissue together, as for example, skin, internal organs, vessels and other tissue, after they have been severed by injury or surgery. The most common method of using sutures to hold tissue together is inserting the suture into the skin by sewing the tissue together with needles. In order to capture the target tissue, however, the physician must insert the needle through the target tissue at least one time in order to cinch the tissue or capture a portion of the tissue to be tied against adjacent tissue. Due to anatomical structures in the human body and geometric constraints of the needle, it is frequently difficult to adequately secure tissue together using conventional sewing techniques.

A suture is typically an elongate flexible filament, but may take a variety as different thread or thread-like structures, including without limitation fibers, lines, wires, and the like. A suture may be a homogeneous or heterogeneous, and may also comprise a single filament or a composite suture, such as a two or more twisted or woven filaments. In addition, a suture may be made from a wide array of absorbable (i.e., metabolized by the body) or non-absorbable materials known in the art.

In recent years, anchors have been used to secure tissue in applications where needle sewing of the tissue is inconvenient, as for example when the target tissue is not easily reached or has single side access. An anchor typically includes a longitudinal body with the distal end of the body having a pointed configuration for insertion into the tissue and a barbed structure for preventing dislodgement or back-up movement of the anchor after insertion into the tissue. Advantageously, anchors can be inserted linearly, with only single side access to the target tissue. The proximal end of a typical surgical anchor (the end opposite the end that is inserted into the tissue) is configured for mechanical attachment to the end of a suture.

A number of different mechanical attachments are used to interconnect and secure a suture to the end of a surgical anchor. Interconnection between the anchor and the suture may be achieved by knotting the suture, by mechanical crimping about the suture, by threading the suture along a tortuous path on the anchor, or other mechanical locking methods. Each of these interconnection methods, however, has associated disadvantages.

In other cases, when it is inconvenient to retrieve the needle after the needle has been passed through the targeted tissue, systems have been developed for placing an anchor on the far side of tissue and using a series of anchors for approximating the tissue. A variety of different techniques and devices have been developed to deliver and attached sutures to tissue. Some techniques involve piercing tissue with needles, tying or forming knots or loops, delivering anchors such as t-tags, x-tags and other flexible or rigid anchors, and the like.

### BRIEF SUMMARY

One example of the invention is a method of forming a surgical fastening system. The method includes the steps of a) placing a portion of an extruded member into a molding cavity of a mold; b) introducing melted plastic into the molding cavity to at least partially encompass a portion of the extruded member; c) permitting the melted plastic to solidify about the extruded member; and d) removing the solidified plastic and encompassed extruded member from the mold. In one exemplary form of the invention, the extruded member is a suture and the mold cavity is configured in the shape of a surgical anchor.

Another example of the invention is a surgical fastener that includes a first component formed of extruded material having a flexible, elongated configuration. A second component is formed of a molded material and is overmolded to and at least partially encompasses a portion of the first component. In one form, the first extruded component is a suture, and the second component is an anchor for the suture with the anchor being overmolded to an end portion of the suture.

In yet another example of the invention, a method is provided for approximating areas of tissue. A first anchor rigidly affixed to a first end of a suture is inserted into a first area of tissue. A second anchor slidably connected to the suture is inserted into a second area of tissue. The second anchor is slid along the suture toward the first anchor to approximate the second area of the tissue to the first area of tissue.

In yet another example of this invention, a suture anchor that is overmolded to the distal end of suture wherein the suture anchor comprises an elongate body having a first end, a second end, and a longitudinal axis extending between the first and second ends. The first end may have a flared geometry. A lateral suture relief is in the elongate body extending from the first end to a longitudinal position intermediate the first and second ends.

In an example of another aspect of the invention the overmolded anchor portion has at least one sharp and angled end that when driven into tissue tends to catch the tissue and rotate into an anchored position.

In another example of one aspect of the invention, the anchor has a controlled frictional interface for interfacing with the surface of a delivery instrument, such as a hollow tube.

### BRIEF DESCRIPTION OF THE DRAWINGS

While the specification concludes with claims which particularly point out and distinctly claim the invention, it is believed the present invention will be better understood from the following description taken in conjunction with the accompanying drawings, in which like reference numbers identify the same elements in which:

FIG. 1 is a perspective view of suture/anchor system including a pair of anchors with a suture extending between the anchors;

FIG. 2a is a schematic view of one-half of a mold for forming a suture/anchor system according to the principles of the invention;

FIG. 2b is a schematic view of the mold half of FIG. 2a with the end of a previously extruded suture placed in the mold cavity;

FIG. 2c is a schematic view of the mold half of FIGS. 2a and 2b with an anchor overmolded onto the suture end;

FIG. 2d is a suture/anchor system removed from a mold following the molding sequence depicted in FIGS. 2a through 2c;

FIG. 3 is a cross-sectional view of a pair of anchors overmolded on both ends of a suture that is being used to secure a balloon catheter located inside a bladder to the pelvic floor;

FIG. 4 is a schematic view of an alternative exemplary embodiment having an anchor overmolded onto one end of a suture with other anchors slidably placed on the suture;

FIGS. 5a through 5c is a schematic representation of a sequence for approximating two tissue areas using the suture/anchor system of FIG. 4;

FIG. 6 is a cross-sectional view of a T-tag style anchor with a suture overmolded onto one end of a suture;

FIG. 7 is a cross-sectional view of the T-tag anchor shown in FIG. 6 in a hollow needle;

FIG. 8 is an elevational view of the T-tag anchor shown in FIG. 6 in a fully deployed position;

FIG. 9 is a schematic top view of the suture/anchor system illustrated in FIG. 6;

FIG. 10 is a schematic top view of an alternative suture/anchor system showing one end of the anchor resiliently radially expanded to provide a controlled frictional interface on the longitudinal surface of the anchor;

FIG. 11 is a schematic top view of an alternative suture/anchor system showing a pair of radially outwardly extending annular rings on the outer surface of the anchor to provide a controlled frictional interface on the longitudinal surface of the anchor;

FIG. 12 is a schematic perspective view of an alternative suture/anchor system showing an acute angle between the end and longitudinal surfaces of the anchor to assist in rotating the anchor following insertion into tissue;

FIG. 13 is a schematic cross-sectional view of the anchor illustrated in FIG. 12;

FIG. 14 is a schematic top view of an alternative suture/anchor system with a resilient radially expanding end portion; and

FIG. 15 is a schematic cross-sectional view of the suture/anchor system shown in FIG. 14 disposed in a hollow needle.

Reference will now be made in detail to certain exemplary embodiments of the invention, examples of which are illustrated in the accompanying drawings.

### DETAILED DESCRIPTION OF EXEMPLARY EMBODIMENTS

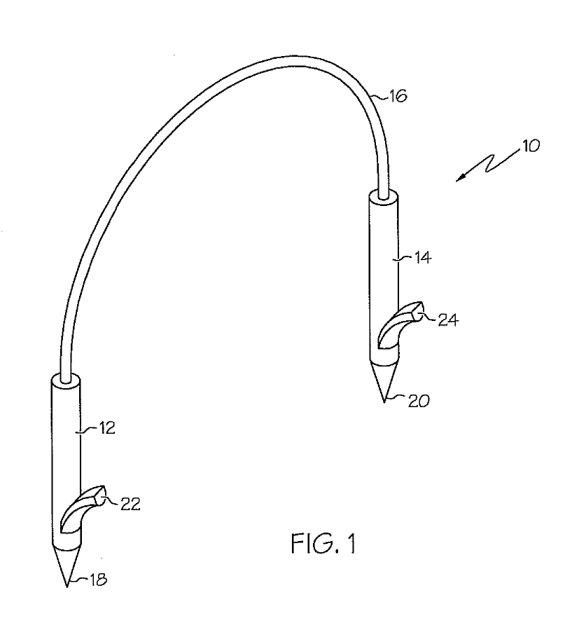

Referring now to the drawings, FIG. 1 shows a simplified form of one exemplary suture/anchor system 10 formed in accordance with the principles of the invention. The system 10 includes first and second surgical anchors, 12 and 14 respectively, commonly joined by a suture 16. The illustrated anchors 12 and 14 each include longitudal bodies of generally cylindrical configuration. The distal (relative to the suture) ends 18 and 20 of the anchors 12 and 14 respectfully are frustoconical in shape with each end portion terminating in a point geometry to facilitate insertion of the anchors 12 and 14 into a tissue. Barbs 22 and 24 extend generally radically outwardly from the bodies 12 and 14 and function to prevent dislodgement or back-up movement of the anchors.

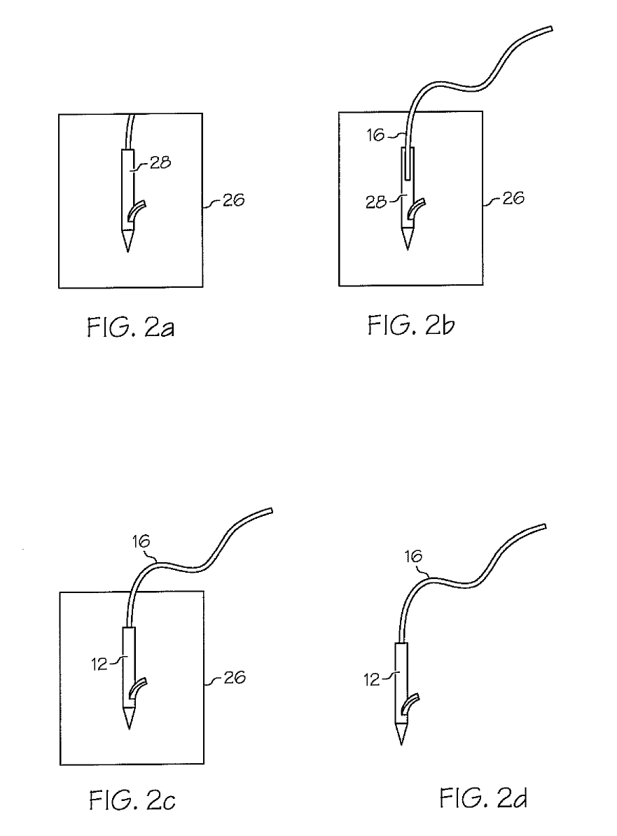

Significantly, the exemplary suture/anchor system 10 shown in FIG. 1 is formed by overmolding the anchors 12 and 14 onto an end of the suture 16. The mold sequence illustrated in FIGS. 2a to 2c schematically shows how the suture/anchor system 10 is formed. Referring specifically to FIG. 2a, one-half of a split mold 26 for forming one end of the suture/anchor system 10 is shown. The mold half 26 has one-half of a split mold cavity 28 into which melted plastic is forced and allowed to cool. As illustrated, the mold cavity 28 has a geometry corresponding to that of the desired geometry of anchors 12 and 14. As schematically illustrated in FIG. 2b, one end of a previously extruded suture 16 is inserted into the portion of the mold cavity 28 prior to the introduction of melted plastic into the cavity 28. Once the end of suture is inserted into the cavity 28, melted plastic is forced into the cavity 28, as schematically shown in FIG. 2c, and the plastic in cavity 28 is permitted to cool and overmold onto the end of suture 16, solidifying into a shape corresponding to the illustrated anchor/suture system 10. As illustrated in FIG. 2d, once the plastic in cavity 28 cools and solidifies, the mold is opened, and the resulting part has a geometry conforming to the shape of the mold cavity 28. Although the mold sequence illustrated in FIGS. 2a through 2c shows only one anchor 12 being molded onto an end of suture 16, it is be readily appreciated that an anchor 14 is similarly overmolded onto the opposite end of suture 16 to form the suture/anchor system 10 illustrated in FIG. 1.

In one exemplary form of the invention, the anchors 12 and 14, as well as the suture 16 are formed of absorbable material, such as for example, a synthetic absorbable copolymer made from 90% glycolide and 10% L-lactide (sold under the trade designation Vicryl) or absorbable polydioxane (PDS). Such absorbable materials are broken down in the tissue over a period of time, typically ranging from 10 days to several weeks, eliminating the need to remove the suture/anchoring system from the patient once the tissue is healed and the suture/anchoring system is no longer necessary. The typical material selected for the suture/anchoring system 10 will be dependent upon the intended use. For example, Vicryl material is highly suitable for general soft tissue approximation and/or ligation, but other materials may be more suitable for other tissue, as for example for cardiovascular and neurological tissues.

Overmolding the anchors onto the suture advantageously includes the option of matching geometries and materials to control the absorbabity profile of the suture/anchoring system 10. In addition to controlling time required for absorption through the geometry of the anchor 12, for example, the absorption profile of the suture/anchor system 10 might be controlled by using different materials for the extruded component (suture) and the molded component (anchor). For example, PDS material (for the anchor) might be overmolded onto a suture formed of PDS, or PDS material (for the anchor) might be overmolded onto a suture formed of Vicryl. Both PDS (polydioxane) and Vicryl (90% glycolide and 10% L-lactide) are absorbable, but the two materials absorb at different rates. Overmolding an anchor 12 onto a suture 16 also permits control of the pullout strength of the interconnection between these two previously separate components. Indeed, it has been found that overmolding onto extruded material, such as a suture, my increase the pull-out force necessary to separate the extruded and molded components relative to conventional methods of attaching such components.

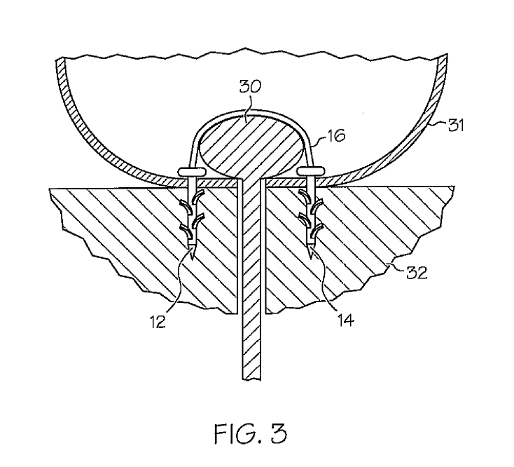

In one exemplary form of the invention, the suture/anchor system 10 allows fixation of tissue at separate points with a specific dimension between the anchors 12 and 14. This allows two the approximation of two separate areas of tissue at a specific distance apart. One exemplary application for a suture/anchoring system with a specific dimension between the two anchors is for a radical prostatectomy procedure where the anchors 12 and 14 are used for insertion through the bladder wall into the pelvic floor for approximating the bladder to the pelvic floor. A suture of a specific length between anchors also may be used to harness a balloon catheter typically used in a harnessed configuration for drainage in a post radical prostatectomy procedure. As illustrated in FIG. 3, two anchors 12 and 14 (having slightly different configurations than the anchors 12 and 14 illustrated in FIG. 1) are overmolded to opposite ends of a suture 16 of specific length to harness a balloon catheter 30 to the pelvic floor 32 of a patient. As shown, the anchors 12 and 14 are inserted through the bladder wall 31 into the pelvic floor 32. This configuration allows the balloon catheter 30 to create downward pressure around the urethra, creating an anastamosis between the bladder and the urethra.

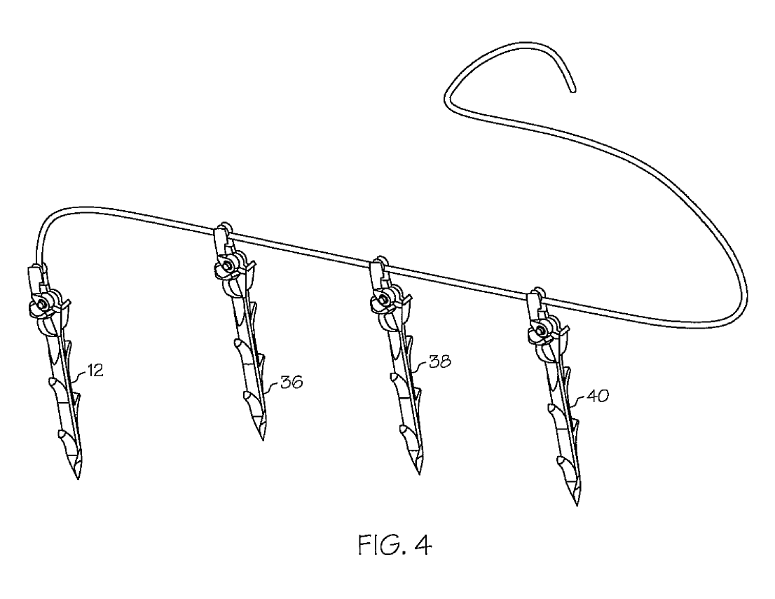

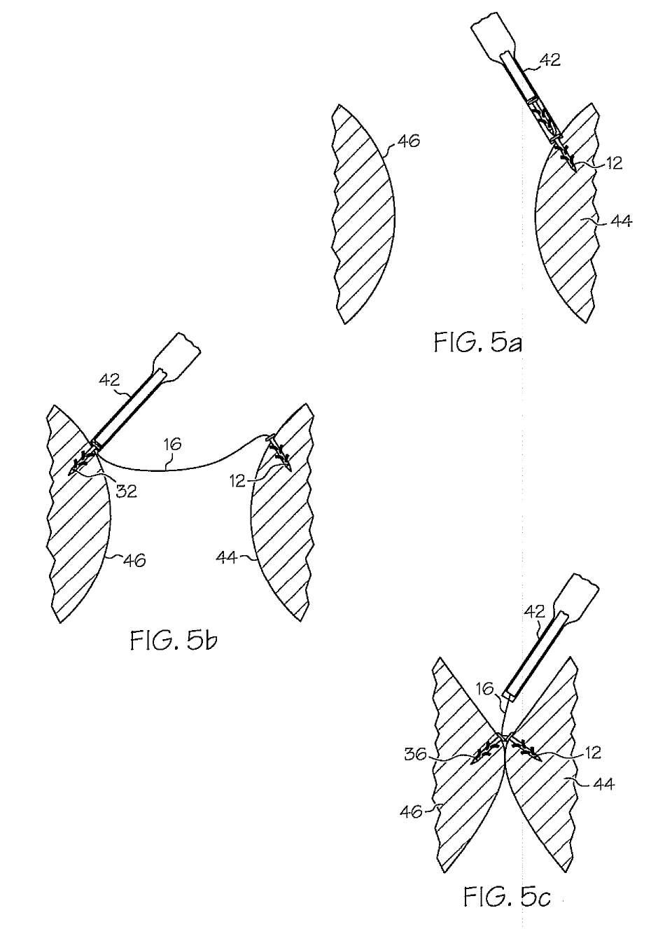

FIG. 4 shows another exemplary embodiment of the invention in which only one anchor 12 (having a different geometry from the anchors illustrated in FIGS. 1 and 3) has been overmolded onto the end of a suture 16. Additional multiple anchors 36, 38 and 40 with slip-through connections to the suture 16 also are shown in FIG. 4. FIGS. 5a through 5c illustrate how the exemplary embodiment of FIG. 4 might be used to approximate two tissue areas. FIG. 5a shows a plunger type anchor applicator 42 linearly inserting an anchor 12 (overmolded onto a suture 16) being linearly inserted into a first tissue area 44. In the illustration of FIG. 5a, the first tissue area 44 is spaced from a second tissue area 46. Turning to FIG. 5b, the anchor applicator 42 inserts a second anchor 36 into the second tissue area 46. Unlike the anchor 12 inserted into tissue area 44, the anchor 36 is not overmolded onto the suture 16, but instead is slidably movable along the suture 16 as a result of its slip-through connection with suture 16. After insertion of the anchor 36 with the slip-through connection into tissue area 46, the applicator 42 draws the portion of the suture 16 opposite from anchor 12, pulling the two anchors together and approximating the tissue areas 44 and 46. Although the sequence illustrated in FIGS. 5a through 5c shows only one overmolded anchor and one anchor with a slip-through connection to suture 16, those skilled in the art will readily appreciate multiple tissue areas might be approximated by inserting multiple anchors (such as anchors 36, 38 and 40) with slid-through connections into multiple tissue areas and pulling the portion of the suture 16 opposite overmolded anchor 12 to create a drawstring-like effect, approximating multiple tissue areas. Optionally, the applicator 42 may be of a type to hold multiple anchors. The applicator 42 also may be designed so that any suture 16 remaining after the last deployed anchor is inserted exits the applicator 42 through a lumen of the applicator and is tied off to draw the anchors (as well as the tissue areas into which the anchors are inserted) into approximation, as illustrated in FIG. 5c.

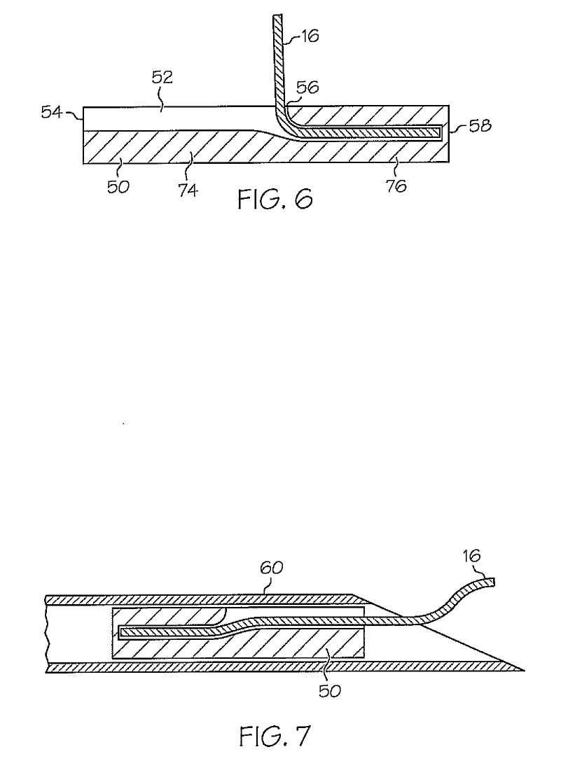

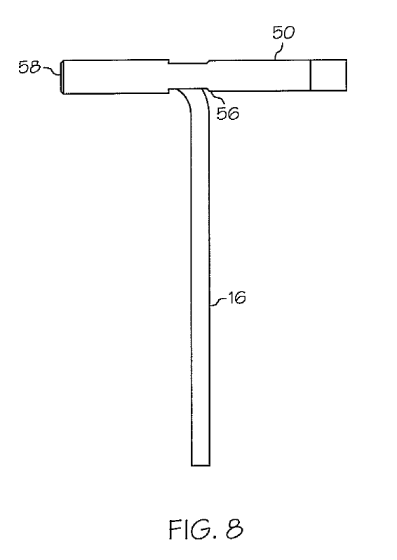

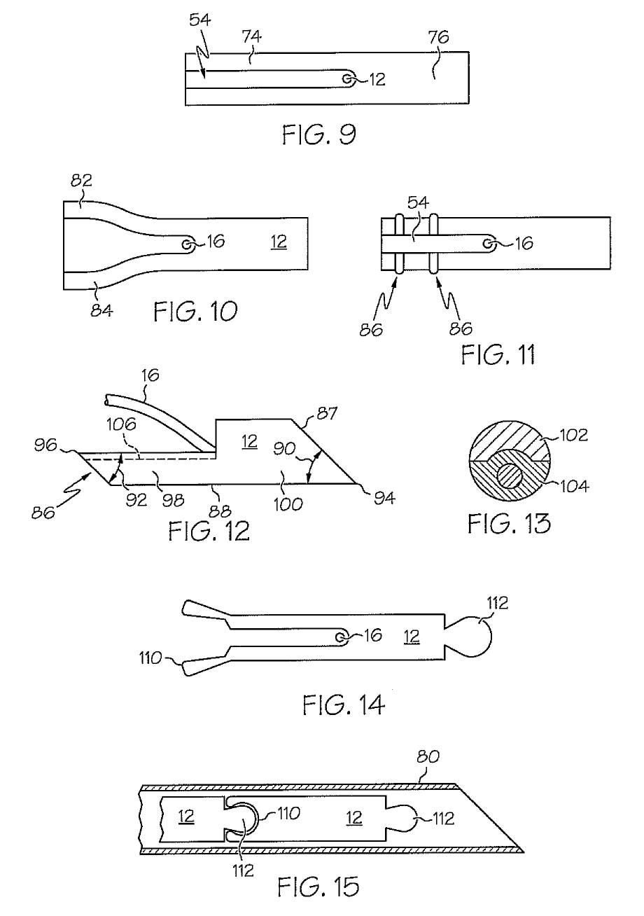

A further exemplary embodiment of the invention is illustrated in FIGS. 6-9. FIG. 6 shows a T-tag type suture/anchor fastening system in which a portion of an anchor body 50 is overmolded onto a suture 16. The anchor body 50 illustrated includes a lateral relief 52, as illustrated in FIGS. 6 and 9, extending from a first end 54 to a longitudinal position intermediate the first end 54 and a second longitudinal end 58. The suture 16 has been overmolded by the body anchor 50 only in the area between the second end 58 and the intermediate position 56. As illustrated in FIG. 7, this exemplary T-tag type anchor has a delivery position in which the suture 16 is generally oriented to be coextensive with the longitudinal axis of the anchor body 50. As the illustration of FIG. 7 suggests, this form of suture/anchor system can be delivered through a hollow needle 60. The deployed position of this T-tag type anchor 50 is illustrated in FIG. 8 wherein the suture 16 extends transverse (generally perpendicular in the drawing) from the longitudinal axis of the anchor body 50 from a location intermediate the ends 54 and 58. It also will be appreciated by those skilled in the art that anchors 50 formed in the T-tag configuration illustrated in FIGS. 6-9 are advantageous in that they can be deployed by passing the needle 60 through the wall of an organ to deploy the anchor 50 in the far wall of the organ. Alternatively, they can be deployed directly into tissue, like the anchors 12 illustrated in FIGS. 1-4.

In the exemplary form of the invention specifically illustrated in FIGS. 6-9, the anchor 12 has a longitudinally surface extending from the first end 72 to the second end 72. This longitudinally extending surface defines first and second longitudinal sections 74 and 76, which in the exemplary embodiment illustrated in FIGS. 6-9, are approximately equal in length. As will be appreciated from a comparison of FIGS. 6 and 7, the relief area 52 permits the suture 16 to freely move relative to the anchor 12 between the delivery position (FIG. 7) (in which the suture 16 is oriented in the longitudinal direction of anchor 12) to a deployed position (FIG. 8)(in which the suture 16 is oriented substantially perpendicular to the anchor's longitudinal direction, as shown in FIG. 6. Optionally, it also may be desirable to provide structure on the longitudinal surface of the anchor 12 to provide a controlled frictional interface between the anchor 12 and the internal surface of the hollow needle 80. One exemplary embodiment for providing such a controlled frictional interface is illustrated in FIG. 10 wherein one end of the anchor 12 includes a plurality of resilient, radially extending members 82 and 84. FIG. 11 shows a still further exemplary alternative structure for controlling the frictional interface between the anchor 12 in the internal surface of the hollow needle 80. In FIG. 11, the anchor 12 includes a plurality of radially extending annular rings or bands 86. As those skilled in the art will appreciate, the annular rings 86 limit the interface surface area between the anchor 12 and the hollow needle 80. By controlling the number of rings 86, the magnitude of friction between the anchor 12 a hollow needle 80 is readily controlled.

FIG. 12 illustrates a still further exemplary embodiment in which the longitudinal ends 86 and 87 of the anchor 12 are obliquely oriented with respect to the longitudinal surface 88. By orienting the surfaces 86 and 87 in this way, acute angles 90 and 92 are formed between the ends 86 and 87 and the longitudinal surface 88. These acute angles 90 and 92 assist in “biting” the tissue and orienting the anchor system in the “Tee” configuration illustrated in FIG. 8. The acute angles 90 and 92 also create sharp edges 94 and 96. The sharp edges 94 and 96 tend to catch tissue and rotate the anchor 12 whenever tension is applied to the suture 16 following insertion of the anchor 12 into tissue, causing rotation of the anchor 12 relative to the suture 16 to move the anchor 12 to the deployed position shown in FIG. 8.

It also will be observed from FIGS. 12 and 13 that the exemplary embodiment disclosed in those figures includes longitudinal sections 98 and 100, which longitudinal sections 98 and 100 have different cross-sectional areas. More specifically, as more clearly shown in FIG. 13, the anchor 12 has a primary cross-section 102 (through longitudinal section 100) which has a cross-sectional area that is greater than the area of the secondary cross-section 104 (through longitudinal 98). Similar to other of the previously disclosed embodiments, the suture 16 in FIGS. 12 and 13 is overmolded only by the longitudinal section 100, and not by the longitudinal section 98. The longitudinal section 98, which has the smaller cross-sectional area, also provides a relief 106 for accommodating a length of the suture 16 when the suture 16 extends in the longitudinal direction of the anchor 12.

It also may be desirable to configure the longitudinal ends of the anchor 12 so that multiple anchors may be stacked in a longitudinal fashion. Referring to FIG. 14, the anchor 12 includes a first end 110 that has a complementary geometry to the opposite end 112 of the anchor 12. This complementary geometry of the first and second ends of the anchor 12 allows the opening 110 of a first anchor 12 to interface with the opposite end 112 of an adjacent anchor 12 to longitudinally stack the adjacent anchors 12 when two anchors 12 are longitudinally aligned as, for example, inside a hollow needle 80, as illustrated in FIG. 15.

The foregoing description of the preferred embodiments of the present invention have been presented for purposes of illustration and description. It is not intended to be exhaustive or to limit the invention to the precise form disclosed. Obvious modifications or variations are possible in light of the above teachings. The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The drawings and preferred embodiments do not and are not intended to limit the ordinary meaning of the claims in their fair and broad interpretation in any way.

What is claimed is:

1. A method of forming a surgical fastening system, comprising the steps of:
a) placing a portion of an extruded member into a molding cavity of a mold;
b) introducing melted plastic into the molding cavity to at least partially encompass a portion of the extruded member;
c) permitting the melted plastic to solidify about the extruded number; and
d) removing the solidified plastic and encompassed extruded member from the mold.

2. A method as recited in claim 1 wherein the extruded member is a suture.

3. A method as recited in claim 2 wherein the mold cavity is configured in the shape of a surgical anchor.

4. A method as recited in claim 3 wherein the extruded member and the plastic introduced into molding cavity or both formed of bioabsorbable material.

5. A method as recited in claim 4 wherein the material from which the extruded member is formed differs from the plastic material introduced into the molding cavity.

6. A method as recited in claim 5 wherein the material from which the extruded member is formed has an absorption rate that differs from the absorption rate of the material introduced into the molding cavity.

7. A method as recited in claim 4 wherein the material from which the extruded member is formed is the same as the plastic material introduced into the molding cavity.

8. A method as recited in claim 3 wherein the configuration of the mold cavity includes a barb.

9. A surgical fastener, comprising:
a) a first component, the first component being formed of extruded material and having a flexible, elongated configuration; and
b) a second component, the second component being formed of a molded material and being overmolded to and at least partially encompassing a portion of the first component.

10. A surgical faster as recited in 9 where in the second component is overmolded to an end portion of the first component.

11. A surgical fastener as recited in claim 10 wherein the first extruded component is a suture, and the second component is an anchor for the suture.

12. A surgical fastener as recited in claim 11 wherein the suture and the anchor are each formed of bioabsorbable material.

13. A surgical fastener as recited in claim 12 wherein the anchor has one absorption rate and the suture has a second absorption rate.

14. A surgical fastener as recited in claim 13 wherein the first and second absorption rates are different.

15. A surgical fastener as recited in claim 11 further including an additional anchor overmolded to a second end portion of the suture, the length of the suture between the anchors been fixed.

16. A surgical fastener as recited in claim 11 further including at least one additional anchor slidably connected to the suture.

17. A surgical fastener as recited in claim 11 wherein the anchor has a configuration that includes at least one barb.

18. Any surgical fastener as recited in claim 11 wherein one end of the anchor is overmolded to the suture and the opposite end of the anchor has a tapered configuration to facilitate insertion into tissue.

19. A method of approximating to areas of tissue, comprising:
a) inserting a first anchor rigidly affixed to a first end of a suture into a first area of tissue;
b) inserting a second anchor slidably connected to the suture into a secondary of tissue; and
c) sliding the second anchor along the suture toward the first anchor to approximate the second area of the tissue to the first area of tissue.

20. A method as recited in claim 19 where sliding of the second anchor toward the first anchor is effectuated by pulling on the suture at a location on the opposite side of the second anchor from the first anchor.

21. A surgical connector, comprising:
a) a suture formed of extruded material;
b) a molded anchor adapted for insertion into tissue, the anchor having a longitudinally extending surface defining first and second longitudinally extending sections with and an end surface at each longitudinal end of the anchor, the first section of the anchor being overmolded onto an end portion of the suture and the second section including an opening for accommodating a length of the suture orientated in the longitudinal direction of the anchor, but also freely permitting the suture length to move relative to the anchor to an orientation substantially parallel to the anchor's longitudinal direction.

22. A surgical connector as recited in claim 21 wherein at least one of the ends is obliquely oriented with respect to the longitudinal surface to form an included acute angle between the end and the longitude surface for catching tissue to assist in rotating the anchor following insertion into tissue.

23. A surgical connector as recited in claim 21 wherein the suture is joined to the anchor proximal to the interface of the first and second longitudinal portions.

24. A surgical connector as recited in claim 21 wherein the first longitudinal section has a primary cross-sectional area and the second longitudinal section has a secondary cross-sectional area that is smaller than the cross-sectional area of the first section, and wherein only the first longitudinal section is overmolded onto the suture.

25. A surgical connector as recited in claim 21 wherein the longitudinally extending surface of the anchor is adapted and configured for insertion into a hollow needle.

26. A surgical connector as recited in claim 25 further including at least one projection extending radially outwardly from at least one of the longitudinal sections of the anchor for providing a controlled frictional interface on the longitudinal surface of the anchor.

27. A surgical connector as recited in claim 26 wherein the protrusion has an annular ring configuration.

28. A surgical connector as recited in claim 21 wherein the opposite longitudinal ends of the anchor have complementary geometries whereby the first end of a first anchor interfaces with a second end of a second anchor to permit stacking of multiple anchors in a longitudinal fashion.

29. A surgical connector as recited in claim 21 wherein at least one end of the anchor has at least one resilient, radially expanding component adapted to provide a controlled frictional interface on the longitudinal surface of the anchor.
